(19) United States
(12) Patent Application Publication
(10) Pub. No.: US 2015/0141440 A1
(43) Pub. Date: May 21, 2015

(54) Pharmaceutical Composition for Headache Relief

(71) Applicant: Ford Jennings Mundy, Madison, MS (US)

(72) Inventor: Ford Jennings Mundy, Madison, MS (US)

(21) Appl. No.: 14/226,560

(22) Filed: Mar. 26, 2014

Related U.S. Application Data
(60) Provisional application No. 61/907,096, filed on Nov. 21, 2013.

(51) Int. Cl. A61K 31/522; A61K 31/167; A61K 31/4152; A61K 31/194

(52) U.S. Cl. CPC A61K 31/522; A61K 31/194; A61K 31/167; A61K 31/4152
USPC 514/263.31; 514/404

(57) ABSTRACT

A pharmaceutical composition for headache relief combines a vasoconstricting agent, a pain relief agent, and a disposition altering agent for a three-pronged approach to headache or migraine relief.

The current application claims a priority to the U.S. Provisional Patent application Ser. No. 61/907,096 filed on Nov. 21, 2013.

### FIELD OF THE INVENTION

The present invention relates generally to a pharmaceutical composition for oral or injectable use. More specifically, a pharmaceutical drug consisting a combination of caffeine, isometheptene mucate, and acetaminophen.

### BACKGROUND OF THE INVENTION

In today's fast-pace, multi-tasking, and high stress world, headaches are a daily nuisance. Studies have been shown that stress levels, sleep deprivation, temperature fluctuations, poor posture, alcohol, hunger and a multitude of other daily occurrences play a major role in the frequency and intensity of headaches. Migraines, cluster headaches, sinus headaches, and tension-type headaches are some of the main types of headaches. In fact, there have been 150 different types of headache categories established. With such a large scale of causes for headaches, it is hard to take all the necessary precautions to avoid them.

Over 45 million Americans, according to the National Headache Foundation, experience chronic headaches. Out of these 45 million, 62% suffer specifically from migraines, which are categorized and defined by moderate to severe pain intensity. While the exact causes for migraines are unknown, one theory suggests abnormal brain activity which results in changes in blood vessels. The majority of the time abnormal brain activity causes constriction, which in turn reduces the oxygen flow to the brain; to fight this phenomenon, the blood vessels dilate in order to overcompensate. This expansion increases the pressure on the nerves coiled around the vessels, causing them to shoot off throbbing pain signals. This causes pain and discomfort that the person experiences. Another major type of headaches is called tension headaches, which is a chronic non-progressive headache that is defined by muscle contractions which cause mild to moderate pain at incremental time periods that dissipates in intensity over a prolonged period of time.

Along with these different types of headaches exists the worldwide known phenomenon known as the hangover. Defined by an excess of alcohol in a person's system causing a plurality of different symptoms to arise once the alcohol has left the system. These include fatigue, unconsciousness, thirst, headaches, nausea, vomiting, poor sleep, shakiness, and mood disturbance such as depression and anxiety. There have been a multitude of “cures” developed for this phenomenon, each tailored only for one or two symptoms. In a same category are cluster headaches, with one of the main causes being cigarettes. These headaches occur around the same time each day and season, this has led scientist to conclude that cluster headaches are tied to the human sleep and wake cycle.

In this fast-pace world people can't afford to take time off to recuperate and wait for headache to go away. Working 40 plus hours a week; running errands on the weekends; and taking care of the family in between, dealing with a headache is the last task on the to do list. A solution is needed that not only masks the pain, but also heals the root physiological cause behind it.

The present invention aims to relieve the human body of the headaches listed above, in a fast, safe, and effective manner.

### BRIEF DESCRIPTION OF THE DRAWINGS

FIG. 1 is a diagram depicting the components of the present invention.

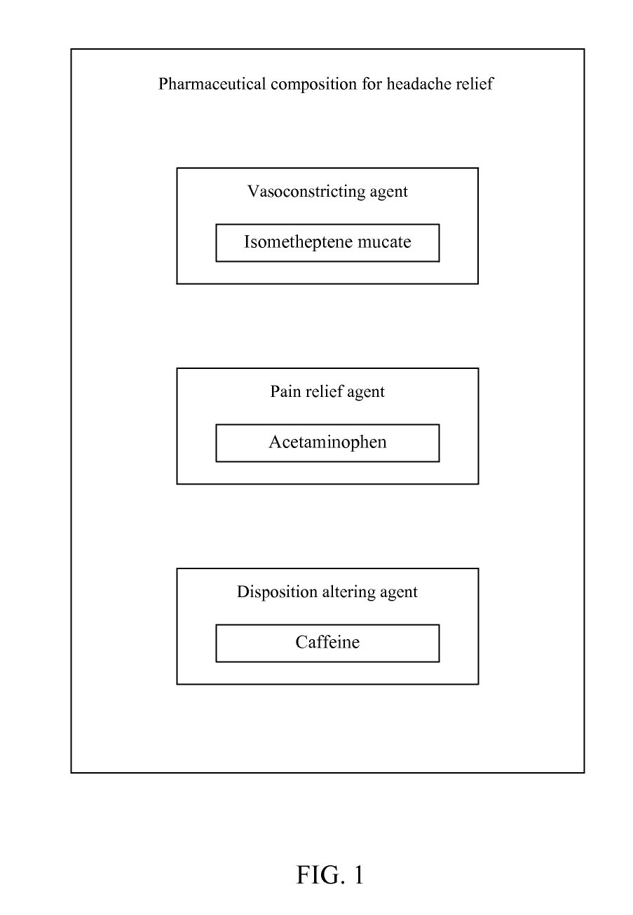

FIG. 2 is a diagram depicting the components of an alternate embodiment of the present invention.

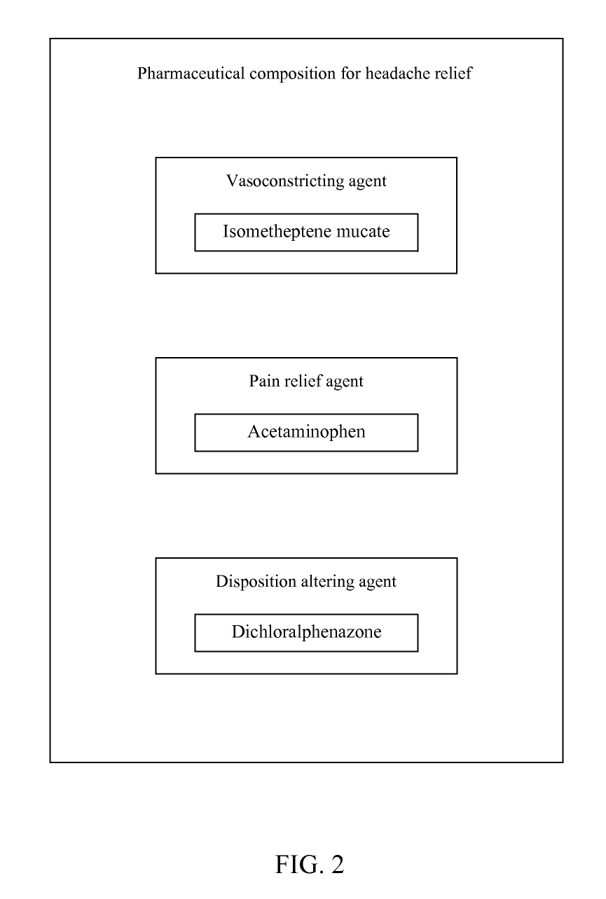

FIG. 3 is a diagram depicting the different ingredients in the preferred embodiment of the present invention.

FIG. 4 is a diagram depicting the different ingredients, and their chemical formula, in the preferred embodiment of the present invention.

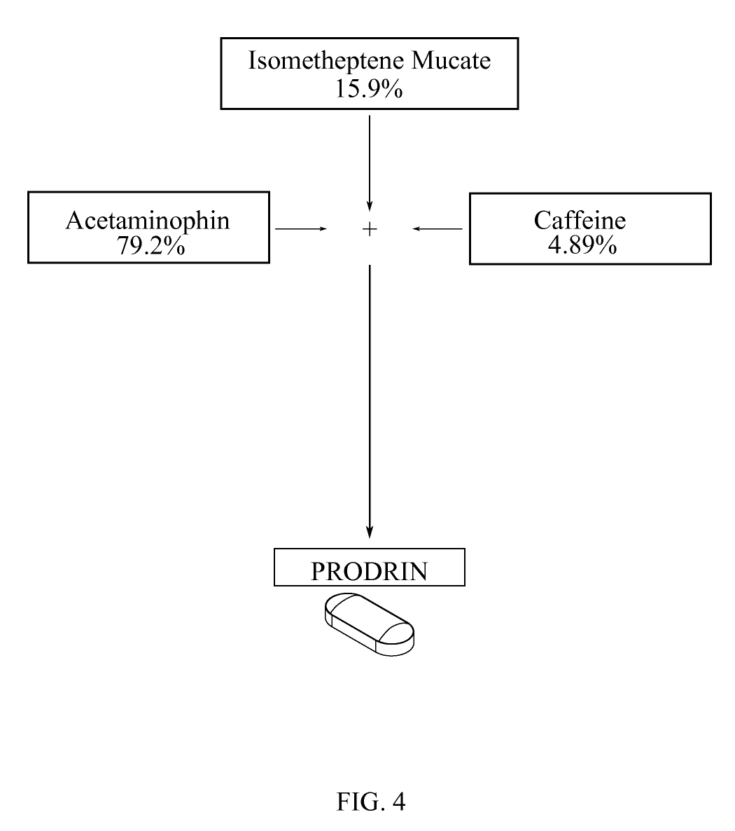

### DETAIL DESCRIPTIONS OF THE INVENTION

All illustrations of the drawings are for the purpose of describing selected versions of the present invention and are not intended to limit the scope of the present invention. The present invention is to be described in detail and is provided in a manner that establishes a thorough understanding of the present invention. There may be aspects of the present invention that may be practiced without the implementation of some features as they are described. It should be understood that some details have not been described in detail in order to not unnecessarily obscure focus of the invention.

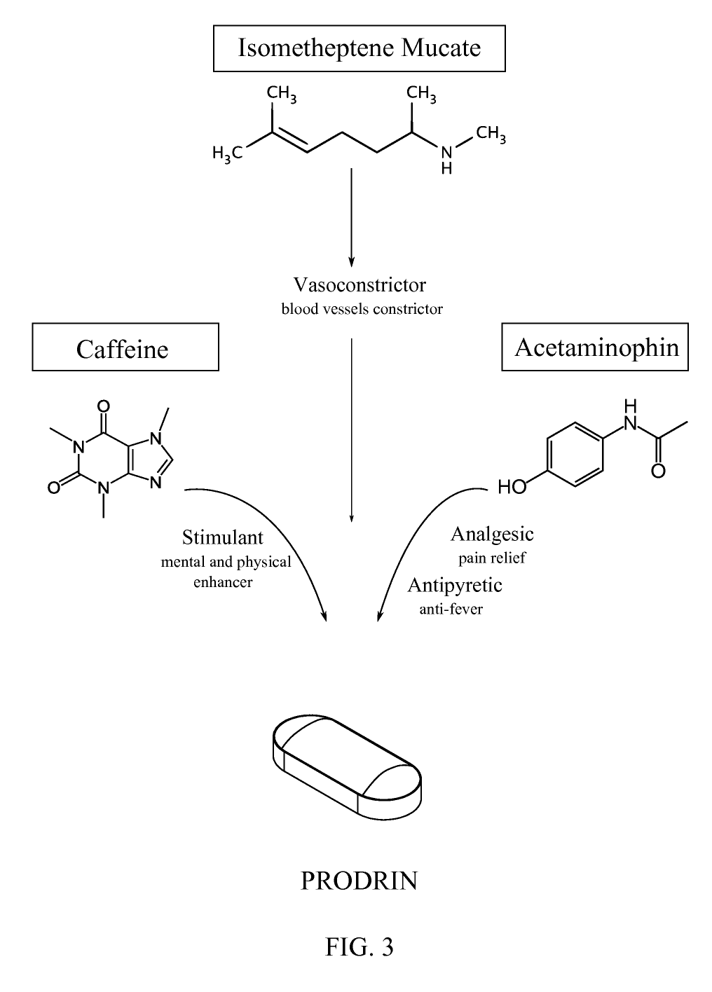

The present invention is a pharmaceutical composition meant to relieve the symptoms of the vast majority of headaches. While the causes of headaches may vary greatly, the majority of symptoms for each headache are similar. Accompanying the throbbing pain is some combination of the following: exhaustion, tiredness, nausea, and depression. The present invention seeks to combat these symptoms through the combination of three different ingredients for three different approaches. The present invention may be referred to by the name Prodrin, as shown in FIG. 3.

In general, the present invention comprises a vasoconstricting agent, a pain relief agent, and a disposition altering agent. The present invention may take any physical form, and as such may be delivered to a user as a powder, a liquid, a pill, or any other appropriate delivery means.

In the preferred embodiment of the present invention, the vasoconstricting agent is isometheptene mucate. The isometheptene mucate is identified with the Chemical Abstracts Service (CAS) registry number of 503-01-5. Isometheptene mucate, also known as simply isometheptene, is a sympathomimetic drug, which means that the drug mimics the effects of transmitter substances of the sympathetic nervous system. Sympathomimetic drugs are typically used to treat cardiac arrest and low blood pressure, or even delay premature labor, among other things. Isometheptene has vasoconstricting properties; that is, it causes constriction of blood vessels and is sometimes used in the treatment of migraines and tension headaches due to said properties. The vasoconstricting agent is present in the preferred embodiment of the present invention at a range between 8-16 percent weight, or at a range between 30-80 milligrams. More specifically, in the preferred embodiment the vasoconstricting agent is present as 65 milligrams of isometheptene mucate, or 15.9 percent weight.

In the preferred embodiment of the present invention, the pain relief agent is acetaminophen. The acetaminophen is identified with the CAS number of 103-90-2. It is contemplated that in alternate embodiments of the present invention the pain relief agent may be aspirin, ibuprofen or another pain relief agent. Acetaminophen, also known as APAP or paracetamol, is a widely used over the counter analgesic (pain reliever) and antipyretic (fever reliever). Acetaminophen is classified as a mild analgesic and is commonly used for the relief of headaches and other minor aches and pains and is a major ingredient in numerous cold and flu remedies. Acetaminophen can also be used to treat inflammatory pain. In the preferred embodiment of the present invention, the pain relief agent is present at a range between 65-90 percent weight, or at a range between 300-325 milligrams. More specifically, in the preferred embodiment the pain relief agent is present as 325 milligrams of acetaminophen, or 79.2 percent weight.

In the preferred embodiment of the present invention, the disposition altering agent is caffeine, identified by the CAS registry number 58-08-2. Caffeine is a stimulant drug commonly consumed by humans in infusions extracted from the seed of the coffee plant and the leaves of the tea bush, among other plant sources. In humans, caffeine acts as a central nervous system stimulant, temporarily warding off drowsiness and restoring alertness, promoting increased wakefulness, faster and clearer flow of thought, increased focus, and better general body coordination. The aforementioned effects serve to aid the user in remedying a headache by providing feelings of improved physical and mental well-being. Additionally, analgesics work more quickly and more efficiently with caffeine, making pain relievers up to 40 percent more effective. Caffeine also aids in the absorption of medications, allowing the patient to feel relief sooner. In the preferred embodiment of the present invention, the disposition altering agent, as caffeine, is present at a range between 2-19 percent weight, or at a range between 10-100 milligrams.

Preferably, the present invention comprises the disposition altering agent as 20 milligrams of caffeine, or 4.89 percent weight.

In an alternate embodiment of the present invention, the disposition altering agent is dichloralphenazone, CAS number 480-30-8. Dichloralphenazone has a different method of action from caffeine as the disposition altering agent, being a relaxant instead of a stimulant. With caffeine, the present invention allows the user to deal with a headache, hangover or other similar ailment while functioning in the context of a work environment, errands or similar activities. With dichloralphenazone, the present invention is more geared toward allowing a user to relax, lie down and let the headache pass. In the alternate embodiment, the disposition altering agent is comprised as 100 milligrams of dichloralphenazone.

Although the invention has been explained in relation to its preferred embodiment, it is to be understood that many other possible modifications and variations can be made without departing from the spirit and scope of the invention as hereinafter claimed.

What is claimed is:

1. A pharmaceutical composition for headache relief comprises:
a vasoconstricting agent;
a pain relief agent;
a disposition altering agent;
the vasocontricting agent being present at a range between 8-16 percent weight;
the pain relief agent being present at a range between 65-90 percent weight; and
the disposition altering agent being present at a range between 2-19 percent weight.

2. The pharmaceutical composition for headache relief as claimed in claim 1, wherein the vasoconstricting agent is isometheptene mucate.

3. The pharmaceutical composition for headache relief as claimed in claim 1, wherein the pain relief agent is acetaminophen.

4. The pharmaceutical composition for headache relief as claimed in claim 1, wherein the disposition altering agent is caffeine.

5. The pharmaceutical composition for headache relief as claimed in claim 1, wherein the disposition altering agent is dichloralphenazone.

6. A pharmaceutical composition for headache relief comprises:
a vasoconstricting agent;
a pain relief agent;
a disposition altering agent;
the vasocontricting agent being present at a range between 30-80 milligrams;
the pain relief agent being present at a range between 300-325 milligrams; and
the disposition altering agent being present at a range between 10-100 milligrams.

7. The pharmaceutical composition for headache relief as claimed in claim 6, wherein the vasoconstricting agent is isometheptene mucate.

8. The pharmaceutical composition for headache relief as claimed in claim 6, wherein the pain relief agent is acetaminophen.

9. The pharmaceutical composition for headache relief as claimed in claim 6, wherein the disposition altering agent is caffeine.

10. The pharmaceutical composition for headache relief as claimed in claim 6, wherein the disposition altering agent is dichloralphenazone.

11. A pharmaceutical composition for headache relief comprises:
a vasoconstricting agent;
a pain relief agent; and
a disposition altering agent.

12. The pharmaceutical composition for headache relief as claimed in claim 11 comprises about 65 milligrams of vasoconstricting agent, wherein the vasoconstricting agent is isometheptene mucate.

13. The pharmaceutical composition for headache relief as claimed in claim 11 comprises about 325 milligrams of pain relief agent, wherein the pain relief agent is acetaminophen.

14. The pharmaceutical composition for headache relief as claimed in claim 11 comprises about 20 milligrams of disposition altering agent, wherein the disposition altering agent is caffeine.

15. The pharmaceutical composition for headache relief as claimed in claim 11 comprises about 100 milligrams of disposition altering agent, wherein the disposition altering agent is dichloralphenazone.
